(19) United States
(12) Patent Application Publication
(10) Pub. No.: US 2009/0312532 A1
(43) Pub. Date: Dec. 17, 2009

(54) Modulation of exon recognition in pre-mrna by interfering with the binding of sr proteins and by interfering with secodary rna structure

(76) Inventors: Judith Christina Theodora Van Deutekom, Dordrecht (NL); Annemieke Aartsma-Rus, Leiden (NL); Garrit-Jan Boudewijn Van Ommen, Amsterdam (NL)

Correspondence Address: TRASKBRITT, P.C., P.O. BOX 2550, SALT LAKE CITY, UT, 84110, US

(21) Appl. No.: 11/919,248

(22) PCT Filed: Apr. 21, 2006
(86) PCT No.: PCT/NL2006/000209

§ 371 Date: Feb. 28, 2008

(30) Foreign Application Priority Data
Apr. 22, 2005 (EP) 05075968.7

(51) Int. Cl. C07H 21/04

(52) U.S. Cl. 536/23.1

(57) ABSTRACT

The invention provides a method for generating an oligonucleotide with which an exon may be skipped in a pre-mRNA and thus excluded from a produced mRNA thereof. Further provided are methods for altering the binding of an SR protein and/or methods for altering the secondary structure of an mRNA to interfere with splicing processes and uses of the oligonucleotides and methods in the treatment of disease. Further provided are pharmaceutical compositions and methods and means for inducing skipping of several exons in a pre-mRNA.

The invention relates to the fields of molecular biology and medicine. More in particular the invention relates to the restructuring of mRNA produced from pre-mRNA, and therapeutic uses thereof.

The central dogma of biology is that genetic information resides in the DNA of a cell and is expressed upon transcription of this information, where after production of the encoded protein follows by the translation machinery of the cell This view of the flow of genetic information has prompted the pre-dominantly DNA based approach for interfering with the protein content of a cell. This view is slowly changing and alternatives for interfering at the DNA level are being pursued.

In higher eukaryotes the genetic information for proteins in the DNA of the cell is encoded in exons which are separated from each other by intronic sequences. These introns are in some cases very long. The transcription machinery generates a pre-mRNA which contains both exons and introns, while the splicing machinery, often already during the production of the pre-mRNA, generates the actual coding region for the protein by splicing together the exons present in the pre-mRNA.

Although much is known about the actual processes involved in the generation of an mRNA from a pre-mRNA, much also remains hidden. In the present invention it has been shown possible to influence the splicing process such that a different mRNA is produced. The process allows for the predictable and reproducible restructuring of mRNA produced by a splicing machinery. An oligonucleotide capable of hybridizing to pre-mRNA at a location of an exon that is normally included in the mature mRNA can direct the exclusion of the thus targeted exon or a part thereof (further referred to as exon-slipping).

The present invention provides alternative methods which are used in the selection process of identifying oligonucleotides suitable for exon skipping processes. The invention further provides oligonucleotides that are, amongst others, capable of skipping exons which could not be skipped before. We had previously identified 37 exon-internal antisense oligonucleotides (AONs) to induce skipping of 14 Duchenne muscular dystrophy (DMD) in human control myotube cultures. We now show new AONs with which we can induce the skipping of a total of 35 exons.

In our WO 2004/083432 patent application we have described a method for generating an oligonucleotide comprising determining, from a (predicted) secondary structure of RNA from an exon, a region that assumes a structure that is hybridised to another part of said RNA (closed structure) and a region that is not hybridised in said structure (open structure), and subsequently generating an oligonucleotide, which at least in part is complementary to said closed structure and which at least in part is complementary to said open structure.

We now disclose an alternative method for designing and generating an oligonucleotide which method can optionally be combined with the method of WO 2004/083432.

We disclose, in the experimental part, the presence of a correlation between the effectivity of an exon-internal antisense oligonucleotide (AON) in inducing exon skipping and the presence of a (for example by ESEfinder) predicted SR binding site in the target pre-mRNA site of said AON. As a result we now show an alternative method for generating an oligonucleotide comprising determining a (putative) binding site for an SR (Ser-Arg) protein in RNA of an exon and producing an oligonucleotide that is complementary to said RNA and that at least partly overlaps said (putative) binding site. The term “at least partly overlaps” is defined herein as to comprise an overlap of only a single nucleotide of an SR binding site as well as multiple nucleotides of said binding site as well as a complete overlap of said binding site. In a preferred embodiment the invention further comprises determining from a secondary structure of said RNA, a region that is hybridised to another part of said RNA (closed structure) and a region that is not hybridised in said structure (open structure), and subsequently generating an oligonucleotide that at least partly overlaps said (putative) binding site and that overlaps at least part of said closed structure and overlaps at least part of said open structure. In this way we increase the chance of obtaining an oligonucleotide that is capable of interfering with the exon inclusion from the pre-mRNA into. mRNA. It is possible that a first selected SR-binding region does not have the requested open-closed structure in which case another (second) SR protein binding site is selected which is then subsequently tested for the presence of an open-closed structure. This process is continued until a sequence is identified which contains an SR protein binding site as well as a(n) (partly overlapping) open-closed structure. This sequence is then used to design an oligonucleotide which is complementary to said sequence.

Such a method for generating an oligonucleotide is also performed by reversing the described order, i.e. first generating an oligonucleotide comprising determining, from a secondary structure of RNA from an exon, a region that assumes a structure that is hybridised to another part of said RNA (closed structure) and a region that is not hybridised in said structure (open structure), and subsequently generating an oligonucleotide, of which at least a part of said oligonucleotide is complementary to said closed structure and of which at least another part of said oligonucleotide is complementary to said open structure. This is then followed by determining whether an SR protein binding site at least overlaps with said open/closed structure. In this way the method of WO 2004/083432 is improved. In yet another embodiment the selections are performed simultaneously.

The term complementarity is used herein to refer to a stretch of nucleic acids that can hybridize to another stretch of nucleic acids under physiological conditions. It is thus not absolutely required that al the bases in the region of complementarity are capable of pairing with bases in the opposing strand. For instance, when designing the oligonucleotide one may want to incorporate for instance a residue that does not base pair with the base on the complementary strand. Mismatches may to some extent be allowed, if under the circumstances in the cell the stretch of nucleotides is capable of hybridising to the complementary part. In a preferred embodiment a complementary part comprises at least 3, and more preferably at least 4 consecutive nucleotides. The complementary regions are preferably designed such that, when combined, they are specific for the exon in the pre-mRNA. Such specificity may be created with various lengths of complementary regions as this depends on the actual sequences in other (pre-)mRNA in the system. The risk that also one or more other pre-mRNA will be able to hybridise to the oligonucleotide decreases with increasing size of the oligonucleotide. It is clear that oligonucleotides comprising mismatches in the region of complementarity but that retain the capacity to hybridise to the targeted region(s) in the pre-mRNA, can be used in the present invention. However, preferably at least the complementary parts do not comprise such mismatches as these typically have a higher efficiency and a higher specificity, than oligonucleotides having such mismatches in one or more complementary regions. It is thought that higher hybridisation strengths, (i.e. increasing number of interactions with the opposing strand) are favourable in increasing the efficiency of the process of interfering with the splicing machinery of the system.

Preferably, the complementarity is between 90 and 100%. In general this allows for approximately 1 or 2 mismatch(es) in an oligonucleotide of around 20 nucleotides.

The secondary (open-closed) structure is best analysed in the context of the pre-mRNA wherein the exon resides. Such structure may be analysed in the actual RNA However, it is currently possible to predict the secondary structure of an RNA molecule (at lowest energy costs) quite well using structure-modelling programs. A non-limiting example of a suitable program is RNA mfold version 3.1 server (Mathews et al 1999, J. Mol. Biol. 288: 911-940). A person skilled in the art will be able to predict, with suitable reproducibility, a likely structure of the exon, given the nucleotide sequence. Best predictions are obtained when providing such modelling programs with both the exon and flanking intron sequences. It is typically not necessary to model the structure of the entire pre-mRNA.

The same is true for the presence or absence of an SR protein binding site. A non-limiting example of a suitable program is ESEfinder.

The open and closed structure to which the oligonucleotide is directed, are preferably adjacent to one another. It is thought that in this way the annealing of the oligonucleotide to the open structure induces opening of the closed structure whereupon annealing progresses into this closed structure. Through this action the previously closed structure assumes a different conformation. The different conformation results in the disruption of the exon inclusion signal. However, when potential (cryptic) splice acceptor and/or donor sequences are present within the targeted exon, occasionally a new exon inclusion signal is generated defining a different (neo) exon, i.e. with a different 5′ end, a different 3′ end, or both. This type of activity is within the scope of the present invention as the targeted exon is excluded from the mRNA. The presence of a new exon, containing part of the targeted exon, in the mRNA does not alter the fact that the targeted exon, as such, is excluded. The inclusion of a neo-exon can be seen as a side effect which occurs only occasionally. There are two possibilities when exon skipping is used to restore (part of) an open reading frame that was disrupted as a result of a mutation. One is that the neo-exon is functional in the restoration of the reading frame, whereas in the other case the reading frame is not restored. When selecting oligonucleotides for restoring reading frames by means of exon-skipping it is of course clear that under these conditions only those oligonucleotides are selected that indeed result in exon-skipping that restores the open reading frame, with or without a neo-exon.

Without wishing to be bound be any theory it is currently thought that use of an oligonucleotide directed to an SR protein binding site results in (at least partly) impairing the binding of an SR protein to the binding site of an SR protein which results in disrupted or impaired splicing.

Preferably, an open/closed structure and an SR protein binding site partly overlap and even more preferred an open/closed structure completely overlaps an SR protein binding site or an SR protein binding site completely overlaps an open/closed structure. This allows for an improved disruption of exon inclusion.

Pre-mRNA can be subject to various splicing events, for instance through alternative splicing. Such events may be induced or catalysed by the environment of a cell or artificial splicing system. Thus, from the same pre-mRNA several different mDRNA's may be produced. The different mRNA's all included exonic sequences, as that is the definition of an exon. However, the fluidity of the mRNA content necessitates a definition of the term exon in the present invention. An exon according to the invention is a sequence present in both the pre-mRNA and mRNA produced thereof, wherein the sequence included in the mRNA is, in the pre-mRNA, flanked on one side (first and last exon) or both sides (any other exon then the first and the last exon) by sequences not present in the mRNA. In principle any mRNA produced from the pre-mRNA qualifies for this definition. However, for the present invention, so-called dominant mRNA's are preferred, i.e. mRNA that makes up at least 5% of the mRNA produced from the pre-mRNA under the set conditions. Human immuno-deficiency virus in particular uses alternative splicing to an extreme. Some very important protein products are produced from mRNA ing up even-less than 5% of the total mRNA produced from said virus. The genomic RNA of retroviruses can be seen as pre-mRNA for any spliced product derived from it. As alternative splicing may vary in different cell types the exons are defined as exons in the context of the splicing conditions used in that system. As a hypothetical example; an mRNA in a muscle cell may contain an exon that as absent in an mRNA produced from the same pre-mRNA in a nerve cell. Similarly, mRNA in a cancer cell may contain an exon not present in mRNA produced from the same mRNA in a normal cell.

Alternative splicing may occur by splicing from the same pre-mRNA. However, alternative splicing may also occur through a mutation in the pre-mRNA for instance generating an additional splice acceptor and/or splice donor sequence. Such alternative splice sequences are often referred to as cryptic splice acceptor/donor sequences. Such cryptic splice sites can result in new exons (neo-exons). Inclusion of neo-exons into produced mRNA can be at least in part prevented using a method of the invention. In case a neo-exon is flanked by a cryptic and a “normal” splice donor/acceptor sequence, the neo-exon encompasses the old (paleo) exon. If in this case the original splice donor/acceptor sequence, for which the cryptic splice donor/acceptor has taken its place, is still present in the pre-mRNA, it is possible to enhance the production of mRNA containing the paleo-exon by interfering with the exon-recognition signal of the neo-exon. This interference can be both in the part of the neo-exon corresponding to the paleo-exon, or the additional part of such neo-exons. This type of exon skipping can be seen as splice correction.

In a preferred embodiment, the generated oligonucleotide is complementary to a consecutive part of between 14 and 60 nucleotides and more preferred said oligonucleotide comprises RNA and even more preferred said oligonucleotide is 2′-O-methyl RNA and has a full-length phosphorothioate backbone. Typical examples of oligonucleotide lengths can be derived from Table 1 and/or 2: 15 to 24 nucleotides. 2′O-methyl RNA is a nucleic acid analogue that is characterized by the exceptional hybridization properties that it imparts with complimentary DNA or RNA as well as, an increased stability against enzymatic degradation compared to natural nucleic acids. Most antisense oligonucleotides currently in clinical development incorporate phosphorothioate backbone modifications, to promote resistance to nucleases while preserving the ability to stimulate cleavage of the mRNA target by ribonuclease (RNase) H.

The exon slipping technique can be used for many different purposes. Preferably, however, exon skipping is used for restructuring mRNA that is produced from pre-mRNA exhibiting undesired splicing in a subject. The restructuring may be used to decrease the amount of protein produced by the cell. This is useful when the cell produces a particular undesired protein. In a preferred embodiment however, restructuring is used to promote the production of a functional protein in a cell i.e. restructuring leads to the generation of a coding region for a functional protein. The latter embodiment is preferably used to restore an open reading frame that was lost as a result of a mutation. Preferred genes comprise a Duchenne muscular dystrophy gene (DMD), a collagen VI alpha 1 gene (COL6A1), a myotubular myopathy 1 gene (MTM1), a dysferlin gene (DYSF), a laminin-alpha 2 gene (LAMA2), an emery-dreyfuss muscular dystrophy gene (FMD), and/or a calpain 3 gene (CAPN3). The invention is further delineated by means of examples drawn from the Duchenne muscular dystrophy (DMD) gene. Although this gene constitutes a particularly preferred gene in the present invention, the invention is not limited to this gene.

Duchenne muscular dystrophy (DMD) and Becker muscular dystrophy (BMD) are both caused by mutations in the DMD gene, that is located on the X chromosome and codes for dystrophin (1-6). DMD has an incidence of 1:3500 newborn males. Patients suffer from progressive muscle weakness, are wheelchair bound before the age of 13 and often die before the third decade of their life (7). The generally milder BMD has an incidence of 1:20,000. BMD patients often remain ambulant for over 40 years and have longer life expectancies when compared to DMD patients (8).

Dystrophin is an essential component of the dystrophin-glycoprotein complex (DGC), which amongst others maintains the membrane stability of muscle fibers (9, 10). Frame-shifting mutations in the DMD gene result in dystrophin deficiency in muscle cells. This is accompanied by reduced levels of other DGC proteins and results in the severe phenotype found in DMD patients (11, 12). Mutations in the DMD gene that keep the reading frame intact, generate shorter, but partly functional dystrophins, associated with the less severe BMD (13, 14).

Despite extensive efforts, no clinically applicable and effective therapy for DM) patients has yet been developed (15), although a delay of the onset and/or progression of disease manifestations can be achieved by glucocorticoid therapy (16). Promising results have recently been reported by us and others on a genetic therapy aimed at restoring the reading frame of the dystrophin pre-mRNA in cells from the mdx mouse model and DM) patients (17-23). By the targeted skipping of a specific exon, a DMD phenotype can be converted into a milder BMD phenotype. The skipping of an exon can be induced by the binding of antisense oligoribonucleotides (AONs) targeting either one or both of the splice sites, or exon-internal sequences. Since an exon will only be included in the mRNA when both the splice sites are recognised by the spliceosome complex, splice sites are obvious targets for AONs. This was shown to be successful, albeit with variable efficacy and efficiency (17, 18, 20, 21).

Besides consensus splice sites sequences, many (if not all) exons contain splicing regulatory sequences such as exonic splicing enhancer (USE) sequences to facilitate the recognition of genuine splice sites by the spliceosome (Cartegni, Chew, and Krainer 285-98). A subgroup of splicing factors, called the SR proteins, can bind to these ESEs and recruit other splicing factors, such as U1 and U2AF to (weakly defined) splice sites. The binding sites of the four most abundant SR proteins (SF2/ASF, SC35, SRp40 and SRp55) have been analyzed in detail and these results are implemented in ESEfinder, a web source that predicts potential binding sites for these SR proteins (Cartegni et al. 3568-71). As disclosed herein the experimental part there is a correlation between the effectiveness of an AON and the presence/absence of an SF2/ASF, SC35 and SRp40 binding site. In a preferred embodiment, the invention thus provides a method as described above, wherein said SR protein is SF2/ASF or SC35 or SRp40. Even more preferred said SR protein binds to mRNA encoding exon 8, 46, 48, 52, 54-56, 58, 60-68 or 71-78 of DM1).

Any oligonucleotide fulfilling the requirements of the invention may be used to induce exon skipping in the DMD gene. The invention provides an oligonucleotide or equivalent thereof obtainable by a method as described above or an oligonucleotide or equivalent thereof capable of inducing exon skipping as depicted in Table 2. The invention further provides an oligonucleotide of Table 2, complementary to exons 8, 46, 48, 52, 54-56, 58, 60-63 or 71-78 of the human DMD gene. An equivalent comprises a similar, preferably the same hybridisation capacity in kind, not necessarily in amount and can for example be a fragment of said oligonucleotide, or an oligonucleotide with a pointmutation, a deletion or even an oligonucleotide with additional nucleotides or any combination thereof.

The complementary oligonucleotide generated through a method of the invention is preferably complementary to a consecutive part of between 13 and 50 nucleotides of said exon RNA. In another embodiment the complementary oligonucleotide generated through a method of the invention is complementary to a consecutive part of between 16 and 50 nucleotides of said exon RNA. Preferably, the oligonucleotide is complementary to a consecutive part of between 13-25 nucleotides of said exon RNA. Preferably between 14 and 25 nucleotides of said exon RNA Different types of nucleic acid may be used to generate the oligonucleotide. Preferably, the oligonucleotide comprises RNA, as RNA/RNA hybrids are very stable. Since one of the aims of the exon skipping technique is to direct splicing in subjects it is preferred that the oligonucleotide RNA comprises a modification providing the RNA with an additional property, for instance resistance to endonucleases and RNaseH, additional hybridisation strength, increased stability (for instance in a bodily fluid), increased or decreased flexibility, reduced toxicity, increased intracellular transport, tissue-specificity, etc. Preferably said modification comprises a 2′-O-methyl-phosphorothioate oligononucleotide modification. Preferably said modification comprises a 2′-O-methyl-phosphorothioate oligodeoxyribonucleotide modification. In one embodiment the invention provides a hybrid oligonucleotide comprising an oligonucleotide comprising a 2′-O-methyl-phosphorothioate oligo(deoxy)ribonucleotide modification and locked nucleic acid. This particular combination comprises better sequence specificity compared to an equivalent consisting of locked nucleic acid, and comprises improved effectivity when compared with an oligonucleotide consisting of 2′-O-methyl-phosphorothioate oligo(deoxy)ribonucleotide modification.

With the advent of nucleic acid mimicking technology it has become possible to generate molecules that have a similar, preferably the same hybridisation characteristics in kind not necessarily in amount as nucleic acid itself. Such equivalents are of course also part of the invention. Examples of such mimics equivalents are peptide nucleic acid, locked nucleic acid and/or a morpholino phosphorodiamidate. Suitable but non-limiting examples of equivalents of oligonucleotides of the invention can be found in (Wahlestedt, C. et al. (2000), Elayadi A. N. & Corey, D. R. (2001), Larsen, H. J., Bentin, T. & Nielsen, P. E. (1999), Braasch, D. A. & Corey, D. R. (2002), Summerton, J. & Weller, D. (1997). Hybrids between one or more of the equivalents among each other and/or together with nucleic acid are of course also part of the invention. In a preferred embodiment an equivalent comprises locked nucleic acid, as locked nucleic acid displays a higher target affinity and reduced toxicity and therefore shows a higher efficiency of exon skipping.

An oligonucleotide of the invention typically does not have to overlap with a splice donor or splice acceptor of the exon.

A transcription system containing a splicing system can be generated in vitro. The art has suitable systems available. However, the need for mRNA restructuring is of course predominantly felt for the manipulation of living cells. Preferably, cells in which a desired effect can be achieved through the restructuring of an mRNA. Preferred mRNA's that are restructured are listed herein above. Preferably, genes active in muscle cells are used in the present invention. Muscle cells (i.e. myotubes) are multinucleated cells in which many but not all muscle cell specific genes are transcribed via long pre-mRNA. Such long pre-mRNA's are preferred for the present invention, as restructuring of mRNA's produced from such long mRNA's is particularly efficient. It is thought, though it need not necessarily be so, that the relatively long time needed to generate the full pre-mRNA aids the efficiency of restructuring using a method or means of the invention, as more time is allowed for the process to proceed. The preferred group of genes of which the mRNA is preferably restructured in a method of the invention comprises: COL6A1 causing Bethlem myopathy, MTM1 causing myotubular myopathy, DYSF (dysferlin causing Miyoshi myopathy and LGMD, LAMA2 (laminin alpha 2) causing Merosin-deficient muscular dystrophy, EMD (emerin) causing Emery-Dreyfuss muscular dystrophy, the DMD gene causing Duchenne muscular dystrophy and Becker muscular dystrophy, and CAPN3 (calpain) causing LGMD2A. Any cell may be used, however, as mentioned, a preferred cell is a cell derived from a DMD patient. Cells can be manipulated in vitro, i.e. outside the subjects body. However, ideally the cells are provided with a restructuring capacity in vivo. Suitable means for providing cells with an oligonucleotide or equivalent thereof of the invention are present in the art. An oligonucleotide of the invention, may be for example be provided to a cell in the form of an expression vector wherein the expression vector encodes a transcript comprising said oligonucleotide. The expression vector is preferably introduced into the cell via a gene delivery vehicle. A preferred delivery vehicle is a viral vector such as an adenoviral vector and more preferably an adeno-associated virus vector. The invention thus also provides such expression vectors and delivery vehicles. It is within the skill of the artisan to design suitable transcripts. Preferred for the invention are PolIII driven transcripts. Preferably in the form of a fusion transcript with an U1 or U7 transcript. Such fusions may be generated as described in references 53 and 54. Improvements in means for providing cells with an oligonucleotide or equivalent thereof, are anticipated considering the progress that has already thus far been achieved. Such future improvements may of course be incorporated to achieve the mentioned effect on restructuring of mRNA using a method of the invention. At present suitable means for delivering an oligonucleotide, equivalent or compound of the invention to a cell in vivo comprise, polyethyleneimine (PEI) or synthetic amphiphils (SAINT-18) suitable for nucleic acid transfections. The amphiphils show increased delivery and reduced toxicity, also when used for in vivo delivery. Preferably compounds mentioned in (\check{S}misterová, J., et al (2001). The synthetic amphiphils preferably used are based upon the easily synthetically available ‘long tailed’ pyridinium head group based materials. Within the large group of amphiphils synthesized, several show a remarkable transfection potential combined with a low toxicity in terms of overall cell survival. The ease of structural modification can be used to allow further modifications and the analysis of their further (in vivo) nucleic acid transfer characteristics and toxicity.

An oligonucleotide or equivalent thereof according to the invention may be used for at least in part altering recognition of said exon in a pre-mRNA. In this embodiment the splicing machinery is at least in part prevented from linking the exon boundaries to the mRNA. The oligonucleotide or equivalent thereof of the invention is at least in part capable of altering exon-recognition in a pre-mRNA. This use is thus also provided in the invention. The prevention of inclusion of a targeted exon in an mRNA is also provided as a use for at least in part stimulating exon skipping in a pre-mRNA. As mentioned above, the targeted exon is not included in the resulting mRNA. However, part of the exon (a neo-exon) may occasionally be retained in the produced mRNA. This sometimes occurs when the targeted exon contains a potential splice acceptor and/or splice donor sequence. In this embodiment the splicing machinery is redirected to utilize a previously not (or underused) splice acceptor/donor sequence, thereby creating a new exon (neo-exon). The neo-exon may have one end in common with the paleo-exon, although this does not always have to be the case. Thus in one aspect an oligonucleotide or equivalent thereof of the invention is used for altering the efficiency with which a splice donor or splice acceptor is used by a splicing machinery.

In yet another embodiment the invention provides use of an oligonucleotide or equivalent thereof according to the invention for the preparation of a medicament.

As mentioned, a preferred gene for restructuring mRNA is the DMD gene. The DMD gene is a large gene, with many different exons. Considering that the gene is located on the X-chromosome, it is mostly boys that are affected, although girls can also be affected by the disease, as they may receive a bad copy of the gene from both parents, or are suffering from a particularly biased inactivation of the functional allele due to a particularly biased X chromosome inactivation in their muscle cells. The protein is encoded by a plurality of exons (79) over a range of at least 2.6 Mb. Defects may occur in any part of the DMD gene. Skipping of a particular exon or particular exons can, very often, result in a restructured mRNA that encodes a shorter than normal but at least partially functional dystrophin protein. A practical problem in the development of a medicament based on exon-skipping technology is the plurality of mutations that may result in a deficiency in functional dystrophin protein in the cell. Despite the fact that already multiple different mutations can be corrected for by the skipping of a single exon, this plurality of mutations, requires the generation of a large number of different pharmaceuticals as for different mutations different exons need to be skipped.

In an even more preferred embodiment multiple (at least two) oligonucleotides according to the invention are used in the preparation of a medicament such that more than one exon can be skipped with a single pharmaceutical. This property is not only practically very useful in that only a limited number of pharmaceuticals need to be generated for treating many different Duchenne or Becker mutations. Another option now open to the person skilled in the art is to select particularly functional restructured dystrophin proteins and produce compounds capable of generating these preferred dystrophin proteins. Such preferred end results are further referred to as mild phenotype dystrophins. The structure of the normal dystrophin protein can be schematically represented as two endpoints having structural function (the beads), which are connected to each other by a long at least partly flexible rod. This rod is shortened in many Becker patients. In a particular preferred embodiment the invention provides a method for treating a DMD patient comprising a mutation as depicted in Table 3, comprising providing said patient with an oligonucleotide effective in inducing exon-skipping of the exon mentioned in the first column, or an equivalent thereof. In a preferred embodiment said oligonucleotide comprises an oligonucleotide effective in inducing exon-skipping mentioned in Table 2, or an equivalent thereof.

In view of the above, the present invention further provides the use of an oligonucleotide, an equivalent thereof or a compound of the invention for the preparation of a medicament. Further provided is a pharmaceutical preparation according to the invention. Said oligonucleotide, or an equivalent thereof of the invention can be used for the preparation of a medicament for the treatment of an inherited disease (for example DMD). Similarly provided is a method for altering the efficiency with which an exon in a pre-mRNA is recognized by a splicing machinery, said pre-mRNA being encoded by a gene comprising at least two exons and at least one intron, said method comprising providing a transcription system comprising said splicing machinery and said gene, with an oligonucleotide, equivalent thereof or a compound according to the invention, wherein said oligonucleotide, equivalent thereof or compound is capable of hybridising to at least one of said exons, and allowing for transcription and splicing to occur in said transcription system. Preferably, said gene comprises at least 3 exons.

An oligonucleotide of the invention, or equivalent thereof, may of course be combined with other methods for interfering with the structure of an mRNA. It is for instance possible to include in a method at least one other oligonucleotide that is complementary to at least one other exon in the pre-mRNA. This can be used to prevent inclusion of two or more exons of a pre-mRNA in mRNA produced from this pre-mRNA. In a preferred embodiment, said at least one other oligonucleotide is an oligonucleotide, or equivalent thereof, generated through a method of the invention. This part of the invention is further referred to as double- or multi-exon skipping. In most cases double-exon skipping results in the exclusion of only the two targeted (complementary) exons from the pre-mRNA. However, in other cases it was found that the targeted exons and the entire region in between said exons in said pre-mRNA were not present in the produced mRNA even when other exons (intervening exons) were present in such region. This multi-skipping was notably so for the combination of oligonucleotides derived from the DMD gene, wherein one oligonucleotide for exon 45 and one oligonucleotide for exon 51 was added to a cell transcribing the DMD gene. Such a set-up resulted in mRNA being produced that did not contain exons 45 to 51. Apparently, the structure of the pre-mRNA in the presence of the mentioned oligonucleotides was such that the splicing machinery was stimulated to connect exons 44 and 52 to each other.

It was found possible to specifically promote the skipping of also the intervening exons by providing a linkage between the two complementary oligonucleotides. To this end the invention provides a compound capable of hybridising to at least two exons in a pre-mRNA encoded by a gene, said compound comprising at least two parts wherein a first part comprises an oligonucleotide having at least 8 consecutive nucleotides that are complementary to a first of said at least two exons, and wherein a second part comprises an oligonucleotide having at least 8 consecutive nucleotides that are complementary to a second exon in said pre-mRNA. The at least two parts are linked in said compound so as to form a single molecule. The linkage may be through any means but is preferably accomplished through a nucleotide linkage. In the latter case the number of nucleotides that not contain an overlap between one or the other complementary exon can be zero, but is preferably between 4 to 40 nucleotides. The linking moiety can be any type of moiety capable of linking oligonucleotides. Currently, many different compounds are available that mimic hybridisation characteristics of oligonucleotides. Such a compound is also suitable for the present invention if such equivalent comprises similar hybridisation characteristics in kind not necessarily in amount. Suitable equivalents were mentioned earlier in this description. One or preferably, more of the oligonucleotides in the compound are generated by a method for generating an oligonucleotide of the present invention. As mentioned, oligonucleotides of the invention do not have to consist of only oligonucleotides that contribute to hybridisation to the targeted exon. There may be additional material and/or nucleotides added. The invention farther provides a composition comprising a first oligonucleotide of the invention capable of hybridising to an exon in a pro-mRNA of a gene or an equivalent of said first oligonucleotide, and at least a second oligonucleotide of the invention capable of hybridising to another exon in a pre-mRNA of a gene or an equivalent of said second oligonucleotide. In a preferred embodiment said first and at least said second oligonucleotide or equivalent thereof are capable of hybridising to different exons on the same pre-mRNA. The composition can be used to induce exon skipping of the respective exons. It has been observed that when the composition comprises oligonucleotides or equivalents thereof directed toward exons 45 and 51, or 42 and 55 of the human DMD gene, that as an exception to the rule that only the targeted exons are excluded from the resulting mRNA, instead the targeted exons and the entire intervening region is excluded from the resulting mRNA. In the present invention this feature is used to correct a variety of different debilitating mutations of the DMD gene. Thus in one embodiment the invention provides a method for the treatment of a subject comprising a mutation in the human DMD gene, wherein as a result of said mutation the DMD gene is not appropriately translated into a functional dystrophin protein, comprising providing said subject with a composition as mentioned above. Mutations that can be corrected in this way are typically mutations that lie within or adjacent to the targeted exon or in the intervening region. However, it is also possible to correct frame-shifting mutations that lie further outside the mentioned exons and intervening region.

The invention will be explained in more detail in the following description, which is not limiting the invention.

### MATERIAL AND METHODS

### AONs, Transfection and RT-PCR Analysis

AON design was based on (Partly) overlapping predicted open secondary structures of the target RNA as predicted by the m-fold program (Mathews et al. 911-40). Some previously described AONs Cable 1) were further analysed by gel mobility shift assays (van Deutekom et al. 1547-54; Aartsma-Rus et al. S71-S77).

All AONs (see Table 1) were synthesized by Eurogentec (Belgium) and contain 2′-O-methyl PNA and full-length phosphorothioate backbones. Myotube cultures derived from a human control were transfected as described previously (van Deutekom et al. 1547-54). Each AON was transfected at least twice at different concentrations (varying from 200 nM to 1 μM with 2 μl-3.5 μl ExGen 500 (MBI Fermentas) per μg AON. A control AON with a 5′ fluorescein label was used to ascertain optimal transfection efficiencies (in general over 90% A). RNA isolation and RT-PCR analysis were performed as described previously (Aartsma-Rus et al. S71-S77), using Transcriptor reverse transcriptase (Roche diagnostics) according to the manufacturer's instructions. PCR primers (Eurogentec, Belgium) were previously described (Aartsma-Rus et al. S71-S77), or chosen in exons flanking the exon targeted by the AONs (sequences upon request).

### Statistical Analysis

Statistical analyses were performed using the R software and the exactRankTests package (R Development Core Team; Hothorn and Hornik). The Wilcoxon signed rank sum test was used to identify significantly higher values when comparing two groups of AONs. The Kruskal Wallis signed rank sum test was performed to determine whether one of three groups was significantly different from the other groups.

### Results

### Efficacy of New AONs

The efficacy of the newly designed series of 77 AONs has not been reported yet. These AONs were tested at least twice at different concentrations and their effectiveness was determined with RT-PCR analysis. The characteristics of all AONs and their efficacies are shown in Table 1. Specific exon skipping, as confirmed by sequence analysis (data not shown), was induced with 61 of the 77 novel AONs (66%) at each of the tested concentrations. For each targeted exon at least one AON was effective, except for exon 47 and exon 57, which remain unskippable.

We further subdivided the AONs that did induce exon slipping into two groups: AONs that induce exon skipping in less than 26% of the transcripts (indicated by a single “plus” in Table 1), and AONs that induce exon skipping in over 25% of the transcripts (indicated by a double “plus”). An example of varying levels of exon 46 skipping is shown in FIG. 1. In total, 25 of the new AONs induced skipping levels of less than 25% and 26 induced skipping levels of over 25%.

The majority of effective AONs induced the skipping of the targeted exon only. Notably, AONs targeting exon 8 always induced the double exon skipping of both exon 8 and the in-frame exon 9 and never single exon 8 skipping (data not shown). In addition to single exon 40, 58 and 73 skipping, AONs targeting exon 40, 58 or 73 occasionally induced low levels of both exon 40 and 41, or 58 and 59 or 73 and 74 skipping, respectively (data not shown). In response to our newly designed exon 51 specific AONs a cryptic splice site in exon 51 was sometimes used, as has been described for previous exon 51 specific AONs (Aartsma-Rus et al. S71-S77; Aartsma-Rus et al. 907-14).

### Evaluation of AONs

We have thus far tested a total of 114 AONs (shown in Table 1); 76 (67% A) of these induced exon skipping (41 in over 25% of transcripts, and 35 in less than 25% of transcripts), whereas 38 (33% A) were ineffective. In order to see whether there is a correlation between efficacy and AON characteristics, we evaluated a number of parameters for groups of effective and ineffective AONs. Potential SR binding sites were calculated for each exon using the ESEfinder software without a threshold. For all binding sites overlapping an AON target site only the highest predicted value for each of the SR proteins is given in Table 1. An example of putative SR binding sites present in exon 46 is shown in FIG. 2 (for clarity only the sites above the standard threshold values provided by the software are shown). Potential SR binding sites that are only partly covered by an AON were not taken into account (for instance AONs 20 and 25 and the second putative SRp40 site in FIG. 2). In addition, the AON lengths, available nucleotides and GC-content were compared (Table 1). The fraction of available nucleotides was determined as the amount of nucleotides targeting an unbound nucleotide in the predicted secondary RNA structure, divided by the length of the AON (FIG. 3).

Boxplots of the effective and ineffective AONs for each of the different variables are depicted in FIG. 4A. Remarkably, the values for SF2/ASF and SC35 as predicted by ESEfinder are clearly higher for the effective AONs than for the ineffective AONs. This difference was statistically significant with p-values <0.1 and <0.05 for SF2/ASF and SC35, respectively, as calculated with the Wilcoxon rank sum test. No significant difference was found for the predicted SRp40 and SRp55 values. The length of the AONs, the fraction of the available nucleotides or the GC-content of the AONs also did not correlate with AON efficacy.

We subdivided the effective AONs into the two subgroups (inducing skipping in either more or less than 25% of the transcript) and made boxplots accordingly (FIG. 4B). No statistically significant difference was observed between the groups for any of the variables using the Kruskal-Waliis signed rank sum test. However, when only two groups were compared using the Wilcoxon rank sum test we did observe a statistical increase for SF2/ASF values in the >26% skipping group when compared to the ineffective group (p-value <0.05) and the <25% skipping group (p-value <0.1). For SC35 values only the difference between the ineffective and <25% skipping group was significant (p-value <0.05). The predicted values of the >25% skipping group were significantly higher than both the ineffective and the <25% skipping group for SRp40 (p-values <0.1). Finally, the GC-content of the >25% skipping group was higher than the ineffective group (p-value <0.1).

Exon skipping can be efficiently induced by AONs targeting either the 5′ splice site or, alternatively, exon internal sequences (Wilton et al. 330-8; Dunckley et al. 1083-90; Mann et al. 42-7; De Angelis et al. 9456-61; Mann et al. 644-54; Lu et al. 6; Goyenvalle et al. 1796-99; Lu et al. 198-203) (Takeshima et al. 515-20; van Deutekom et al. 1547-54; Takeshima et al. 788-90; Aartsma-Rus et al. S71-S77; Aartsma-Rus et al. 907-14; Aartsma-Rus et al. 83-92; Takeshima). However, exon-internal AONs may have some advantages over splice site AONs. First, exon-internal AONs are generally more specific, since they target the coding sequence and not the splice sites, which are partly determined by a consensus sequence. This may not hold true for every splice site. For instance, the 5′ splice site of the murine DMD exon 23 targeted in most exon skipping studies in the mdx mouse, differs to a great extent from the consensus splice site. However, on average at least part of a splice site specific AON will consist of consensus sequences, potentially allowing the adverse targeting of other exons. Furthermore, Mann and colleagues concluded that the most important variable in splice site AON design is the target sequence Mann et al. 644-54; Mann et al. 42-7). Finding an effective 5′ splice site AON for the murine exon 23 required extensive optimisation (Mann et al. 644-54). In contrast, the design of exon-internal AONs has proved to be rather straightforward, since there is a larger window of target sequence. On average two out of three DMD AONs targeting an open structure in the predicted pre-mRNA are effective. This is underscored by the observation that 76 out of the 114 exon-internal AONs described in this study are effective, and together induce the skipping of in total 35 of the 37 targeted DMD exons.

Since ESEfinder has recently become publicly available we analysed in whether our AONs target predicted SF2/ASF, SC36, SRp40 or SRp55 binding sites. Interestingly, we observed significantly higher values for the two most abundant SR proteins (i.e. SF2/ASF and SC35) for effective AONs when compared to ineffective AONs, whereas we did not observe significant differences for predicted SRp40 and SRp65 binding sites. However, when we subdivided the effective AONs into efficient AONs (<25% skipping) and very efficient AONs (>26% skipping), we did observe significantly higher values for the very efficient group for SRp40 values when compared to the inefficient and efficient groups. It should be noted that not every effective AON has high values for these SR proteins, while some ineffective AONs do have high values. One should bear in mind, however, that ESEfinder values reflect a prediction for putative ESE sites. Nevertheless, there seems to be a significant trend towards higher SR values for effective AONs, and we will thus design our future AONs primarily to predicted SF2/ASF, SC35 and SRp40 binding sites. On comparison, no significant difference was observed between effective and ineffective AONs for the length, the fraction of available nucleotides or the GC-content. Nonetheless, the GC-content of AONs, which induce skipping levels of over 25%, is significantly higher than that of ineffective AONs. This may be explained by the fact that AONs with a higher GC-content have a higher melting temperature, and are therefore more likely to bind to their target RNA.

The fact that 67% of our AONs is effective is impressive and may be due to the complexity of the DMD gene that is 2.4 Mb long and contains introns that are generally over 30 kb long. Thus, the splicing of this gene may already be problematic and rely more on exonic splicing enhancer sequences than other genes. Furthermore, the occurrence of some extremely large introns (>100 kb) makes it unlikely that the gene is consecutively spliced. Intron 7 for example is 110 kb long, whereas intron 8 is only 1113 bp. Since we and others (Dr. Steve Wilton, personal communication; Luis Garcia, personal communication) observe only the simultaneous skipping of both exon 8 and 9 after single exon 8 targeting, it is likely that in the vast majority of DMD transcripts intron 8 is spliced out prior to the huge intron 7. Similarly, for AONs targeting exons 40, 58 and 73 we sometimes observed the skipping of an adjacent exon in addition to the skipping of the targeted exon. This is an indication that in a fraction of the transcripts the distal intron is spliced out prior to the proximal one and may suggest the delayed splicing of these introns.

Since there is a wide spectrum of mutation for DMD patients, AONs specific for many individual internal exons (i.e. exon 2-63 and 71-78) will be required. Since exons 64-70 code for the cysteine rich region, which is essential for dystrophin functionality, restoring the reading frame in this area will not result in a functional proteins. Designing DMD exon-internal AONs using the predicted secondary pre-mRNA target structure and/or the predicted presence/absence of an SR protein binding site (preferably, SF2/ASF or SC35) is remarkably effective.

### Tables

TABLE 1
Characteristics of used AONs

ESEfinder value over

| | | |
|---|---|---|
| Targeted | threshold² | Fraction |

| | | | | | | | | | | |
|---|---|---|---|---|---|---|---|---|---|---|
| AON | Sequence | exon | Skip¹ | SF2/ASF | SC35 | SRp40 | SRp55 | Length | open³ | % GC |

| | | | | | | | | | | |
|---|---|---|---|---|---|---|---|---|---|---|
| h2AON1⁵ | | 2 | ++ | 1.49 | 1.54 | 3.37 | 1.12 | 24 | 0.29 | 29% |
| h2AON2⁵ | | 2 | − | 1.49 | 1.44 | 1.40 | 2.71 | 22 | 0.32 | 36% |
| h2AON3⁶ | gaaaauugugcauuuacccauuuu | 2 | − | 1.69 | 1.44 | 1.40 | 2.71 | 24 | 0.29 | 29% |
| h8AON1 | cuuccuggauggcuucaau | 8 | ++ | 1.31 | 0.12 | 2.57 | 2.67 | 19 | 0.53 | 47% |
| h8AON3 | guacauuaagauggacuuc | 8 | ++ | −1.19 | 0.70 | 1.82 | 3.22 | 19 | 0.53 | 37% |
| h17AON1 | ccauuacaguugucuguguu | 17 | ++ | 3.77 | 2.92 | 3.04 | 2.91 | 20 | 0.40 | 40% |
| h17AON2 | uaaucugccucuucuuuugg | 17 | + | 1.76 | −0.68 | 3.83 | 1.54 | 20 | 0.60 | 40% |
| h19AON⁴ | ucugcuggcaucuugc | 19 | + | 2.83 | 1.92 | 2.28 | 2.46 | 16 | 0.58 | 58% |
| h29AON1⁵ | | 29 | ++ | 5.74 | 1.07 | 4.60 | 3.53 | 20 | 0.30 | 45% |
| h29AON2⁵ | | 29 | ++ | 5.74 | 1.07 | 4.60 | 2.04 | 20 | 0.40 | 60% |
| h29AON4 | ccaucuguuagggucugug | 29 | ++ | 3.09 | 3.24 | 2.40 | 2.91 | 19 | 0.58 | 53% |
| h29AON6 | ucugugccaauaugcgaauc | 29 | ++ | 1.26 | 3.28 | 2.33 | 4.33 | 20 | 0.55 | 45% |
| h29AON9 | uuaaaugucucaaguucc | 29 | + | 1.83 | 1.41 | 1.09 | 1.39 | 18 | 0.28 | 33% |
| h29AON10 | guaguucccuccaacg | 29 | − | 1.61 | 0.79 | 1.68 | −0.11 | 16 | 0.44 | 56% |
| h29AON11 | cauguaguucccucc | 29 | + | 0.13 | 1.95 | 3.63 | 3.16 | 15 | 0.67 | 53% |
| h40AON1⁶ | | 40 | ++ | 1.31 | −0.39 | 1.44 | 0.77 | 19 | 0.56 | 37% |
| h40AON2⁶ | | 40 | ++ | 2.81 | 2.78 | 3.93 | 1.21 | 19 | 0.47 | 53% |
| h41AON1⁶ | | 41 | ++ | 3.82 | −0.39 | 1.63 | 0.93 | 19 | 0.74 | 47% |
| h41AON2⁶ | | 41 | + | 2.39 | 2.62 | 1.32 | 0.66 | 20 | 0.50 | 35% |
| h42AON1⁶ | | 42 | + | 2.89 | 3.20 | 5.75 | 3.14 | 17 | 0.47 | 47% |
| h42AON2⁶ | | 42 | + | 3.23 | 3.37 | 1.98 | 1.19 | 18 | 0.00 | 50% |
| h43AON1⁶ | | 43 | − | 1.83 | 1.47 | 3.61 | 2.83 | 18 | 0.39 | 50% |
| h43AON2⁶ | | 43 | + | −0.78 | 1.08 | −0.24 | 0.10 | 19 | 0.63 | 26% |
| h43AON3 | uguuaacuuuuucccauugg | 43 | − | 0.50 | 1.06 | 4.15 | 0.10 | 20 | 0.55 | 35% |
| h43AON4 | cauuuuguuaacuuuuuccc | 43 | − | −0.78 | 1.06 | 1.11 | 0.06 | 20 | 0.45 | 30% |
| h43AON5⁷ | | 43 | ++ | 1.37 | 2.97 | 1.43 | 2.57 | 19 | 0.37 | 63% |
| h44AON1⁷ | | 44 | ++ | 0.25 | 0.64 | 0.88 | 2.51 | 19 | 0.26 | 58% |
| h44AON2⁷ | | 44 | ++ | −0.64 | 1.47 | 2.01 | 2.41 | 20 | 0.40 | 35% |
| h45AON1⁶ | | 45 | − | 1.79 | 1.01 | 3.07 | 2.41 | 19 | 0.37 | 42% |
| h45AON2⁶ | | 45 | − | 3.03 | 0.82 | 2.07 | 0.93 | 19 | 0.74 | 42% |
| h45AON3 | ucuguuuuugaggauugc | 45 | − | 0.37 | 1.82 | 1.97 | 1.35 | 18 | 0.39 | 39% |
| h45AON4 | ccaccgcagauucaggc | 46 | − | 3.27 | 1.45 | 1.81 | 3.39 | 17 | 0.47 | 65% |
| h45AON5⁷ | | 45 | + | 0.50 | 2.30 | 1.19 | 0.35 | 17 | 0.29 | 65% |
| h45AON9 | uuugcagaccuccugcc | 45 | − | 3.98 | 3.20 | 0.86 | 2.56 | 17 | 0.65 | 59% |
| h46AON4⁵ | | 46 | + | 2.34 | 2.82 | 1.68 | 0.01 | 15 | 0.07 60% | |
| h46AON6⁵ | | 46 | + | 2.34 | 2.82 | 1.68 | 2.45 | 20 | 0.15 | 50% |
| h46AON8⁵ | | 46 | ++ | −1.14 | 1.08 | 3.52 | 1.04 | 20 | 0.60 | 40% |
| h46AON9⁵ | | 46 | − | 0.66 | 1.30 | 0.51 | 2.83 | 15 | 1.00 | 40% |
| h46AON20 | gaaauucugacaagauauucu | 46 | + | 1.35 | 1.08 | 2.07 | 1.48 | 21 | 0.48 | 29% |
| h46AON21 | uaaaacaaauucauu | 46 | − | −2.28 | −0.40 | −0.72 | 0.83 | 15 | 0.40 | 13% |
| h46AON22 | uccagguucaagugggauac | 46 | ++ | 2.39 | 3.47 | 3.70 | 0.78 | 20 | 0.60 | 50% |
| h46AON23 | uuccagguucaagug | 46 | ++ | 1.61 | 1.03 | 1.47 | 0.78 | 15 | 0.53 | 47% |
| h46AON24 | ucaagcuuuucuuuuag | 46 | + | −1.19 | −1.09 | 3.52 | 0.18 | 17 | 0.35 | 29% |
| h46AON25 | cugacaagauauucuu | 46 | + | −0.80 | 1.08 | 0.74 | 1.48 | 16 | 0.88 | 31% |
| h46AON26 | agguucaagugggauacua | 46 | ++ | 2.39 | 3.47 | 3.70 | 2.09 | 19 | 0.79 | 42% |
| h47AON1⁵ | | 47 | − | 3.82 | 1.56 | 3.68 | 1.21 | 16 | 0.22 | 50% |
| h47AON2⁶ | | 47 | − | −0.69 | 2.17 | 2.20 | 0.53 | 21 | 0.48 | 29% |
| h47AON3 | uccaguuucuuuaauuguuug | 47 | − | 1.70 | 0.22 | 2.76 | 1.02 | 22 | 0.45 | 27% |
| h47AON4 | cugcuugagcuuauuuucaaguu | 47 | − | 0.74 | 2.17 | 2.20 | 0.53 | 23 | 0.39 | 35% |
| h47AON5 | agcacuuacaagcacgggu | 47 | − | −1.37 | 2.05 | 1.25 | 2.07 | 19 | 0.53 | 53% |
| h47AON8 | uucaaguuuaucuugcucuuc | 47 | − | 1.11 | 0.96 | 0.74 | −0.40 | 21 | 0.33 | 33% |
| h48AON1⁵ | | 48 | − | 0.83 | 0.08 | 2.44 | 1.38 | 18 | 0.81 | 38% |
| h48AON2⁶ | | 48 | − | 0.64 | 1.50 | 2.33 | 1.31 | 21 | 0.48 | 24% |
| h48AON3 | ggucuuuuauuugagcuuc | 48 | − | 0.01 | 1.72 | 2.83 | 1.58 | 19 | 0.74 | 37% |
| h48AON4 | cuucaagcuuuuuuucaagcu | 48 | − | −1.34 | 1.32 | 2.32 | 0.42 | 21 | 0.62 | 33% |
| h48AON8 | gcuucaauuucuccuuguu | 48 | + | 0.63 | 0.34 | 1.62 | 2.57 | 19 | .0.63 | 37% |
| h48AON7 | uuuauuugagcuucaauuu | 48 | + | 0.01 | 1.72 | 1.62 | 2.57 | 19 | 0.68 | 21% |
| h48AON8 | gcugcccaaggucuuuu | 48 | − | 0.91 | 1.98 | 0.25 | 1.90 | 17 | 0.53 | 53% |
| h48AON9 | cuucaaggucuucaagcuuuu | 48 | + | 0.91 | 1.98 | 2.32 | 2.21 | 21 | 0.62 | 38% |
| h48AON10 | uaacugcucuucaaggucuuc | 48 | + | 0.91 | 1.98 | 2.32 | 2.21 | 21 | 0.48 | 43% |
| h49AON1⁶ | | 49 | ++ | 3.02 | 0.52 | 1.96 | 3.41 | 19 | 0.42 | 47% |
| h49AON2⁵ | | 49 | ++ | 0.56 | 0.05 | 0.70 | 1.38 | 19 | 0.32 | 47% |
| h50AON1⁶ | | 50 | ++ | 1.69 | 3.02 | 2.71 | −0.03 | 17 | 0.24 | 47% |
| h50AON2⁶ | | 50 | + | 1.10 | 1.37 | 1.41 | 2.83 | 15 | 0.47 | 67% |
| h51AON1⁵ | | 51 | ++ | −0.31 | 1.48 | 1.35 | 0.41 | 20 | 0.70 | 40% |
| h51AON24 | gaaagccagucgguaaguuc | 51 | − | 1.77 | 1.14 | 4.90 | 2.04 | 20 | 0.60 | 50% |
| h51AON27 | cacccaccaucaccc | 51 | − | 0.39 | 1.74 | 0.38 | 1.31 | 15 | 0.00 | 67% |
| h51AON2⁵ | | 51 | ++ | 2.68 | 2.27 | 3.94 | 2.91 | 23 | 0.22 | 30% |
| h51AON29 | ugauauccucaaggucaccc | 51 | ++ | 1.67 | 1.91 | 2.88 | 2.82 | 20 | 0.25 | 50% |
| h52AON1 | uugcuggucuuguuuuuc | 52 | + | 1.58 | 3.61 | 2.44 | 0.52 | 18 | 0.50 | 39% |
| h52AON2 | ccguaaugauuguucu | 52 | − | −0.07 | 1.11 | 2.23 | −0.80 | 18 | 0.25 | 38% |
| h53AON1⁶ | | 53 | + | 3.08 | 2.28 | 1.83 | 0.77 | 18 | 0.78 | 61% |
| h53AON2⁵ | | 53 | − | 2.20 | 4.04 | 3.40 | 0.21 | 18 | 0.50 | 61% |
| h54AON1 | uacauuugucugccacugg | 54 | ++ | 3.77 | 1.64 | 4.00 | 1.88 | 18 | 0.65 | 50% |
| h54AON2 | cccggagaaguuucaggg | 54 | ++ | 3.14 | 1.80 | 3.54 | 1.34 | 19 | 0.68 | 58% |
| h55AON1 | cuguugcaguaaucuaugag | 55 | + | 0.74 | 4.82 | 4.92 | 2.92 | 20 | 0.65 | 40% |
| h55AON2 | ugccauuguuucaucagcucuuu | 55 | + | 2.70 | 2.29 | 3.46 | 1.27 | 23 | 0.52 | 39% |
| h55AON3 | ugcaguaaucuaugaguuuc | 55 | + | 0.74 | 4.82 | 4.92 | 2.41 | 20 | 0.60 | 35% |
| h55AON5 | uccuguaggacauuggcagu | 55 | ++ | 3.03 | 2.67 | 5.88 | 2.34 | 20 | 0.35 | 50% |
| h55AON6 | gagucuucuaggagccuu | 55 | ++ | 0.87 | 5.77 | 3.38 | 0.33 | 18 | 0.28 | 50% |
| h56AON1 | uuuuuuggcuguuuucaucc | 56 | + | 2.77 | 1.66 | 2.52 | 2.22 | 20 | 0.55 | 35% |
| h56AON2 | guucacuccacuugaaguuc | 56 | − | 0.78 | 1.88 | 4.04 | 1.52 | 20 | 0.35 | 45% |
| h56AON3 | ccuuccagggaucucagg | 56 | + | 1.81 | 5.52 | 3.88 | 0.27 | 18 | 0.58 | 61% |
| h57AON1 | uaggugccugccggcuu | 57 | − | 2.11 | 3.30 | 2.54 | 2.03 | 17 | 0.41 | 65% |
| h57AON2 | cugaacugcuggaaagucgcc | 57 | − | 2.47 | 1.98 | 2.77 | 2.41 | 21 | 0.57 | 57% |
| h57AON3 | uucagcuguagccacacc | 57 | − | 2.83 | 4.73 | 4.81 | 4.10 | 18 | 0.28 | 56% |
| h58AON1 | uucuuuaguuuucaauucccuc | 58 | − | 0.63 | 1.70 | 2.52 | 1.60 | 22 | 0.64 | 32% |
| h58AON2 | gaguuucucuaguccuucc | 58 | + | 1.65 | 3.48 | 2.18 | 0.68 | 16 | 0.37 | 47% |
| h59AON1 | caauuuuucccacucaguauu | 59 | − | 1.77 | 0.34 | 3.53 | 2.23 | 21 | 0.57 | 33% |
| h59AON2 | uugaaguuccuggagucuu | 59 | ++ | 1.31 | 4.84 | 3.28 | 1.34 | 19 | 0.47 | 42% |
| h60AON1 | guucucuuucagaggcgc | 60 | + | 0.66 | 3.86 | 2.29 | 3.00 | 18 | 0.58 | 56% |
| h60AON2 | gugcugagguuauacggug | 60 | − | 2.87 | 2.56 | 4.08 | 2.78 | 19 | 0.84 | 53% |
| h61AON1 | gucccugugggcuucaug | 61 | − | 5.26 | 2.92 | 5.97 | 2.57 | 19 | 0.37 | 58% |
| h61AON2 | gugcugagaugcuggacc | 61 | + | 2.28 | 3.32 | 4.43 | 3.64 | 18 | 0.66 | 61% |
| h62AON1 | uggcucucccaggg | 62 | ++ | 1.08 | 0.33 | 1.69 | −0.50 | 16 | 0.50 | 69% |
| h62AON2 | gggcacuuuguuuggcg | 62 | − | 1.70 | 0.56 | 1.71 | 0.09 | 17 | 0.47 | 59% |
| h63AON1 | ggucccagcaaguuguuug | 63 | + | 1.70 | 0.97 | 3.18 | 1.25 | 19 | 0.79 | 53% |
| h63AON2 | guagagcucugucauuuuggg | 63 | + | 2.61 | 2.57 | 3.12 | 0.93 | 21 | 0.38 | 48% |
| h71AON1 | gccagaaguugaucagagu | 71 | ++ | 0.12 | 3.35 | 4.36 | 1.47 | 18 | 0.79 | 47% |
| h71AON2 | ucuacuggccagaaguug | 71 | ++ | 1.37 | 4.61 | 4.36 | 1.47 | 18 | 0.50 | 50% |
| h72AON1 | ugaguaucaucgugugaaag | 72 | ++ | 6.59 | 0.60 | 8.02 | 0.25 | 20 | 0.60 | 40% |
| h72AON2 | gcauaauguucaaugcgug | 72 | + | 0.77 | 2.43 | 1.26 | 2.14 | 19 | 0.47 | 42% |
| h73AON1 | gauccauugcuguuuucc | 73 | ++ | 1.22 | 0.89 | 2.16 | 2.47 | 18 | 0.39 | 44% |
| h73AON2 | gagaugcuaucauuuagauaa | 73 | + | −0.48 | 0.68 | 2.26 | 3.64 | 21 | 0.29 | 29% |
| h74AON1 | cuggcucaggggggagu | 74 | ++ | 1.35 | 2.39 | 2.35 | 1.39 | 17 | 0.59 | 71% |
| h74AON2 | uccccucuuuccucacucu | 74 | + | 3.04 | 0.33 | 1.68 | 2.82 | 19 | 0.16 | 53% |
| h75AON1 | ccuuuauguucgugcugcu | 75 | ++ | 3.64 | 1.41 | 3.39 | 2.83 | 19 | 0.21 | 47% |
| h75AON2 | ggcggccuuuguguugac | 75 | ++ | 1.51 | 1.11 | 3.71 | 1.12 | 18 | 0.39 | 61% |
| h76AON1 | gagagguagaaggagagga | 76 | − | 0.08 | 1.28 | 3.53 | 3.22 | 19 | 0.32 | 53% |
| h76AON2 | auaggcugacugcugucgg | 76 | + | 3.23 | 1.47 | 4.30 | 1.58 | 19 | 0.32 | 58% |
| h77AON1 | uuguguccuggggagga | 77 | ++ | 4.28 | 3.50 | 3.57 | −0.18 | 17 | 0.47 | 59% |
| h77AON2 | ugcuccaucaccuccucu | 77 | ++ | 2.43 | 0.32 | −0.21 | 1.65 | 18 | 0.39 | 56% |
| h78AON1 | gcuuuccagggguauuuc | 78 | ++ | 1.81 | 4.04 | 3.32 | 0.62 | 18 | 0.78 | 60% |
| h78AON2 | cauuggcuuuccagggg | 78 | ++ | 1.81 | 2.95 | 3.32 | 0.27 | 17 | 0.71 | 59% |

¹++ Exon skipping detected in over 25% transcripts in normal control myotube cultures; + exon skipping detected in up to 25% of transcripts; − no exon skipping detected
²For each AON the highest value is gives for each of the SR proteins
³The fraction of available nucleotides targeted by the AON in the predicted secondary RNA structure over the total length of the AON
⁴This AON targets part of the ESE deleted in the deletion Kobe (Matsuo et al. 963-7; Matsuo et al. 2127-31)
⁶Previously published (van Deutekom et al. 1547-64)
⁶Previously published (Aartsma-Rus et al. S71.)
⁷Previously published (Aartsma-Rus et al. 83-92)

TABLE 2
Selection of novel AONs

ESEfinder values over

| | | |
|---|---|---|
| Targeted | threshold² | Fraction |

| | | | | | | | | | | |
|---|---|---|---|---|---|---|---|---|---|---|
| AON | Sequence | exon | Skip¹ | SF2/ASF | SC35 | SRp40 | SRp55 | Length | open³ | % GC |

| | | | | | | | | | | |
|---|---|---|---|---|---|---|---|---|---|---|
| h8AON1 | cuuccuggauggcuucaau | 8 | ++ | 1.31 | 0.12 | 2.57 | 2.57 | 19 | 0.53 | 47% |
| h8AON3 | guacauuaagauggacuuc | 8 | ++ | −1.19 | 0.70 | 1.82 | 3.22 | 19 | 0.53 | 37% |
| h46AON20 | gaaauucugacaagauauucu | 45 | + | 1.36 | 1.08 | 2.07 | 1.48 | 21 | 0.46 | 29% |
| h46AON22 | uccagguucaagugggauac | 45 | ++ | 2.39 | 3.47 | 3.70 | 0.78 | 20 | 0.80 | 50% |
| h46AON23 | uuccagguucaagug | 46 | ++ | 1.61 | 1.03 | 1.47 | 0.78 | 15 | 0.53 | 47% |
| h46A0N24 | ucaagcuuuucuuuuag | 46 | + | −1.19 | −1.09 | 3.52 | 0.18 | 17 | 0.35 | 29% |
| h46AON25 | cugacaagauauucuu | 48 | + | −0.80 | 1.08 | 0.74 | 1.48 | 16 | 0.88 | 31% |
| h48AON9 | cuucaaggucuucaagcuuuu | 48 | + | 0.91 | 1.98 | 2.32 | 2.21 | 21 | 0.82 | 38% |
| h46AON10 | uaaacugcucuucaaggucuuc | 48 | + | 0.91 | 1.98 | 2.32 | 2.21 | 21 | 0.48 | 43% |
| h52AON1 | uugcuggucuuguuuuuc | 52 | + | 1.66 | 3.81 | 2.44 | 0.52 | 18 | 0.50 | 39% |
| h54AON1 | uacauuugucugccacugg | 54 | ++ | 3.77 | 1.84 | 4.00 | 1.86 | 18 | 0.56 | 50% |
| h54AON2 | cccggagaaguuucaggg | 54 | ++ | 3.14 | 1.80 | 3.54 | 1.34 | 19 | 0.58 | 58% |
| h55AON1 | cuguugcaguaaucuaugag | 55 | + | 0.74 | 4.82 | 4.92 | 2.92 | 20 | 0.65 | 40% |
| h55AON2 | ugccauuguuucaucagcucuuu | 55 | + | 2.70 | 2.29 | 3.48 | 1.27 | 23 | 0.52 | 39% |
| h55AON3 | ugcaguaaucuaugaguuuc | 55 | + | 0.74 | 4.82 | 4.92 | 2.41 | 20 | 0.60 | 35% |
| h55AON5 | uccuguaggacauuggcagu | 56 | ++ | 3.03 | 2.67 | 5.66 | 2.34 | 20 | 0.35 | 50% |
| h55AON6 | gagucuucuaggagccuu | 56 | ++ | 0.87 | 5.77 | 3.36 | 0.33 | 18 | 0.28 | 50% |
| h56AON1 | uuuuuuggcuguuuucaucc | 56 | + | 2.77 | 1.56 | 2.52 | 2.22 | 20 | 0.55 | 35% |
| h56AON3 | ccuuccagggaucucagg | 56 | + | 1.81 | 5.52 | 3.66 | 0.27 | 18 | 0.56 | 61% |
| h58AON2 | gaguuucucuaguccuucc | 58 | + | 1.66 | 3.45 | 2.18 | 0.68 | 19 | 0.37 | 47% |
| h60AON1 | guucucuuucagaggcgc | 60 | + | 0.66 | 3.66 | 2.29 | 3.00 | 18 | 0.58 | 56% |
| h61AON2 | gugcugagaugcuggacc | 61 | + | 2.28 | 3.32 | 4.43 | 3.64 | 18 | 0.58 | 61% |
| h62AON1 | uggcucucccaggg | 62 | ++ | 1.08 | 0.33 | 1.89 | −0.50 | 16 | 0.50 | 89% |
| h63AON1 | ggucccagcaaguuguuug | 63 | + | 1.70 | 0.97 | 3.18 | 1.25 | 19 | 0.79 | 53% |
| h63AON2 | guagagcucugucauuuuggg | 63 | + | 2.81 | 2.57 | 3.12 | 0.93 | 21 | 0.38 | 48% |
| h71AON1 | gccagaaguugaucagagu | 71 | ++ | 0.12 | 3.35 | 4.38 | 1.47 | 19 | 0.79 | 47% |
| h71AON2 | ucuacuggccagaaguug | 71 | ++ | 1.37 | 4.81 | 4.38 | 1.47 | 18 | 0.50 | 50% |
| h72AON1 | ugaguaucaucgugugaaag | 72 | ++ | 6.59 | 0.80 | 6.02 | 0.25 | 20 | 0.60 | 40% |
| h72AON2 | gcauaauguucaaugcgug | 72 | + | 0.77 | 2.43 | 1.28 | 2.14 | 19 | 0.47 | 42% |
| h73AON1 | gauccauugcuguuuucc | 73 | ++ | 1.22 | 0.89 | 2.16 | 2.47 | 18 | 0.39 | 44% |
| h73AON2 | gagaugcuaucauuuagauaa | 73 | + | −0.48 | 0.88 | 2.28 | 3.84 | 21 | 0.29 | 29% |
| h74AON1 | cuggcucaggggggagu | 74 | ++ | 1.35 | 2.39 | 2.35 | 1.39 | 17 | 0.59 | 71% |
| h74AON2 | uccccucuuuccucacucu | 74 | + | 3.04 | 0.33 | 1.88 | 2.82 | 19 | 0.16 | 53% |
| h75AON1 | ccuuuauguucgugcugcu | 75 | ++ | 3.84 | 1.41 | 3.39 | 2.83 | 19 | 0.21 | 47% |
| h75AON2 | ggcggccuuuguguugac | 75 | ++ | 1.51 | 1.11 | 3.71 | 1.12 | 18 | 0.39 | 61% |
| h76AON2 | auaggcugacugcugucgg | 76 | + | 3.23 | 1.47 | 4.30 | 1.58 | 19 | 0.32 | 58% |
| h77AON1 | uuguguccuggggagga | 77 | ++ | 4.28 | 3.50 | 3.57 | −0.18 | 17 | 0.47 | 59% |
| h77AON2 | ugcuccaucaccuccucu | 77 | ++ | 2.43 | 0.32 | −0.21 | 1.65 | 18 | 0.39 | 56% |
| h78AON1 | gcuuuccagggguauuuc | 78 | ++ | 1.81 | 4.04 | 3.32 | 0.62 | 18 | 0.78 | 50% |
| h78AON2 | cauuggcuuuccagggg | 78 | ++ | 1.81 | 2.95 | 3.32 | 0.27 | 17 | 0.71 | 59% |

¹++ Exon skipping detected in over 25% of transcripts in normal control myotube cultures; + exon skipping detected in up to 25% of transcripts; − no exon skipping detected
²For each AON the highest value is gives for each of the SR proteins
³The fraction of available nucleotides targeted by the AON in the predicted secondary RNA structure over the total length of the AON
⁴This AON targets part of the ESE deleted in the deletion Kobe (Matsuo et al. 983-7; Matsuo et al. 2127-31)

TABLE 3
Overview of the mutations for which the reading frame can
currently be restored by AON-induced single exon skipping

| Exon | Applicable to | | | % all |
|---|---|---|---|---|
| to | | | Point | DMD |
| skip¹ | Deletions | Duplications | mutations | mutations |
| 2 | 3-7; | 2 | | 1.4% |
| 8 | 3-7; 4-7; 5-7; 6-7 | 8; 8-9² | | 1.9% |
| 17 | 18; 18-20; 18-25; 18-27; 18-33; 18- | 17 | | 0.6% |
| | 41; 18-44 | | | |
| 19 | 20; 20-27; 20-29 | | | 0.1% |
| 29 | | | 29 | 0.2% |
| 40 | | | 40 | 0.1% |
| 41 | | | 41 | 0.3% |
| 42 | | | 42 | 0.1% |
| 43 | 44; 44-47; 44-48; 44-49; 44-51; | 43 | | 2.9% |
| 44 | 3-43; 5-43; 6-43; 10-43; 13-43; 14- | 44 | | 5.8% |
| | 43; 17-43; 28-43; 30-43; 35-43; 36- | | | |
| | 43; 38-43; 40-43; 42-43; 43; 45; 45- | | | |
| | 54; 46-68 | | | |
| 45 | 44; 46; 46-47; 46-48; 46-49; 46-51; | 45 | | 7.4% |
| | 46-53; 46-55; 46-60 | | | |
| 46 | 21-45; 43-45; 45; 47-54; 47-56 | | | 4.3% |
| 48 | | 48 | 48 | 0.5% |
| 49 | | | 49 | 0.1% |
| 50 | 51; 51-53; 51-55; 51-57 | 50 | | 4.1% |
| 51 | 13-50; 29-50; 43-50; 45-50; 47-50; | 51 | | 9.7% |
| | 48-50; 49-50; 50; 52; 52-63 | | | |
| 52 | 51; 53; 53-55; 53-59; 53-60 | 52 | | 3.9% |
| 53 | 10-52; 43-52; 45-52; 47-52; 48-52; | 53 | | 6.0% |
| | 49-52; 50-52; 52 | | | |
| 54 | 44-53; 46-53; 55 | 54 | | 0.6% |
| 55 | 45-54; 47-54; 48-54; 49-54; 52-54; | | | 1.6% |
| | 54; 56 | | | |
| 56 | 46-55; 55; 57; 57-60 | 56 | | 0.4% |
| 58 | 51-57 | | | 0.04% |
| 59 | | | | 0% |
| 60 | | | 60 | 0.2% |
| 61 | | 61 | | 0.04% |
| 62 | | | | 0% |
| 63 | | | | 0% |
| 71 | | | | 0% |
| 72 | | | 72 | 0.04% |
| 73 | | | | 0% |
| 74 | | | 74 | 0.4% |
| 75 | 65-74 | | | 0.04% |
| 76 | | | | 0% |
| 77 | | | 77 | 0.04% |
| 78 | | | | 0% |

¹Only the exons which have been shown skippable are shown
²AONs targeting exon 8 also induce skipping of the in frame exon 9, implying that these AONs can also be applied to restore the cDNA for a patient with an exon 8-9 duplication

### DESCRIPTION OF FIGURES

FIG. 1. A representative and comparative analysis of effective vs. ineffective AONs. RT-PCR analysis of dystrophin mRNA fragments of control myotube cultures treated with different exon 46 AONs. Clear exon skipping levels of over 25% of the total transcript can be observed for AONs 8, 22, 23 and 26 (indicated by a double plus). AONs 4, 6, 20, 24 and 26 induce skipping levels of less than 26% (indicated by a single plus), where AONs 6 and 24 induce very faint skips. No skipping was observed after treatment with AONs 9 and 21 (indicated by a minus).

FIG. 2. Graphical overview of exon 46 and exon 46 specific AONs. The sequence of exon 46 is depicted with the location of the AONs indicated by lines. The location and values above the thresholds as predicted by ESEfinder for SF2/ASF, SC35 and SRp40 and SRp55 are shown as bars. The threshold values for each of the SR protein as given in ESEfinder as shown between brackets. The most efficient AONs (# 8, 22, 23 and 26) indeed cover putative ESEsites, whereas the ineffective AON # 25 does not completely overlap putative ESEsites. However, the ineffective AON #9 targets potential SRp40 and SRp55 binding sites as well.

FIG. 3. Example of the secondary pre-mRNA structure of exon 46 and flanking sequences as predicted by m-fold. The locations of the 3′ and 65′ splice sites are indicated. The secondary structure consists of dosed structures, in which the nucleotides are bound to other nucleotides within the target RNA, and open structures that consist of unbound nucleotides. The locations of two exon 46 specific AONs are shown (i.e. #6 and #26); the 20-mer #6 targets 3 unbound nucleotides, thus the fraction of available basepairs is 3/20 (0.15). AON #26 is a 19-mer and 15 of its nucleotides target an unbound nucleotide, and thus the fraction of available basepairs is 15/19 (0.79).

FIG. 4. Boxplots of the different groups of AONs for the predicted values of SF2/ASF, SC35, SRp40 and SRp55, and AON length, the fraction of available nucleotides and GC content. A) Comparison of effective vs. ineffective AONs. The values for SF2/ASF and SC35 are significantly higher for the effective AONs than for the ineffective AONs (Wilcoxon rank sum test). No significant difference was observed for the other variables. B) Comparison of ineffective AONs vs. AONs that induce skipping in less than 25% of the transcripts (<25%), and vs. AONs that induce skipping in over than 25% of the transcripts (>25%). No individual group was significantly different from the other groups for any of the variables (Kruskal-Wallis signed rank sum test). When only two of the groups were compared to each other the SF2/ASF and the SRp40 values in the >25% group were significantly higher than those of both the ineffective and <25% group; the <25% group contained significantly higher values than the ineffective group for SC35 and the GC-contained of the >25% group was significantly higher than the ineffective group.

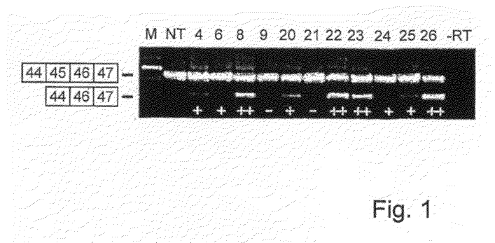

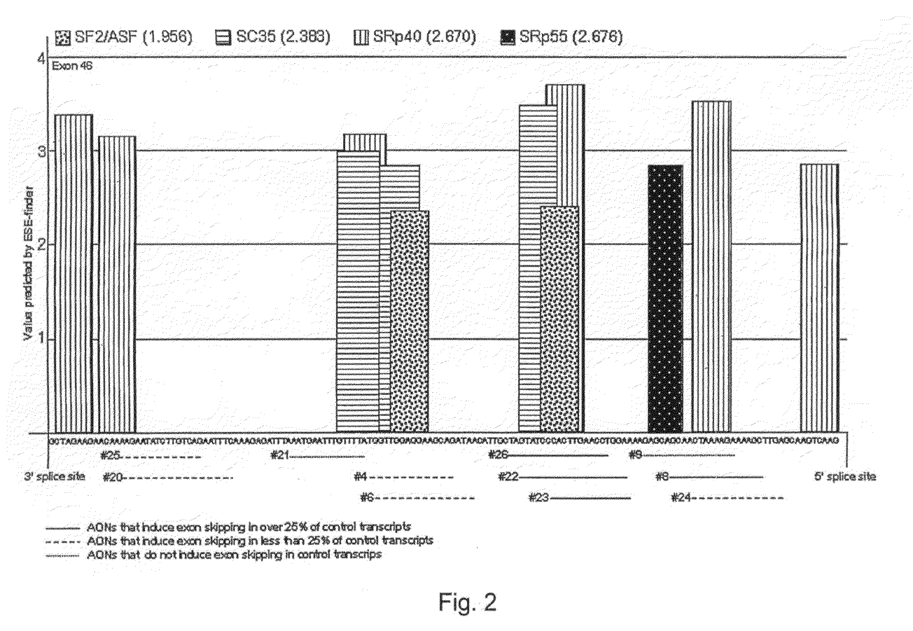

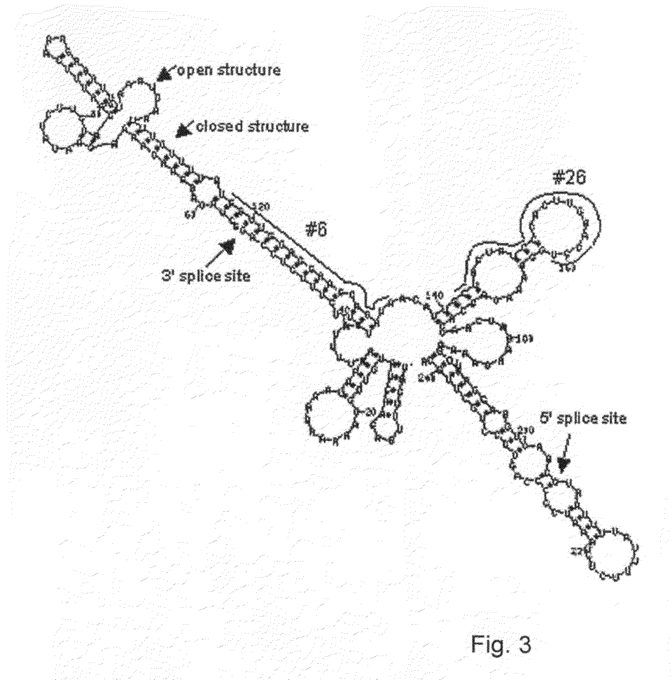

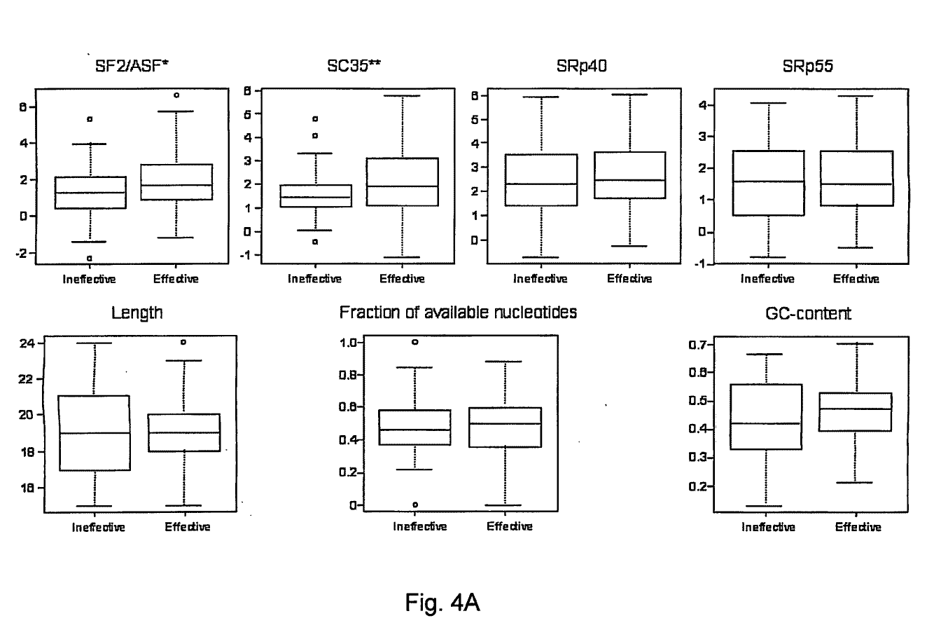

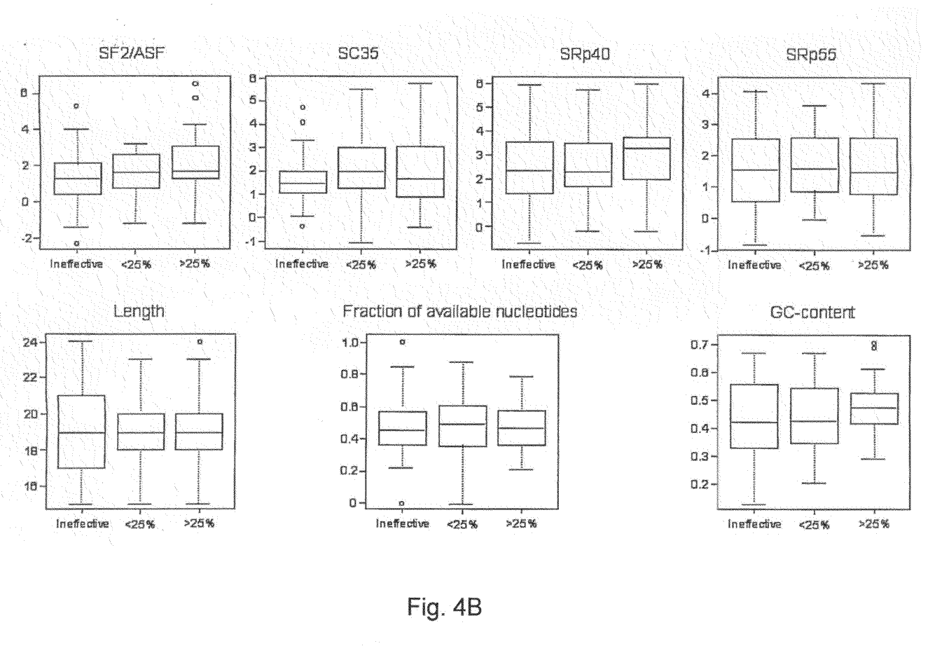

* Difference between the groups is significant with a p-value <0.1, ** p-value -<0.05.

### REFERENCES

1. Hoffman, E. P., Brown, R. H., Jr., Kunkel, L. M. (1987) Dystrophin: the protein product of the Duchenne muscular dystrophy locus. Cell, 51, 919-928.
2. Hoffman, E. P., Fischbeck, K. H., Brown, R. H., Johnson, M., Medori, R., Loike, J. D., Harms, J. B., Waterston, R., Brooke, M., Specht, L., et al. (1988) Characterization of dystrophin in muscle-biopsy specimens from patients with Duchenne's or Becker's muscular dystrophy. N. Engl. J. Med., 318, 1363-1368.
3. Den Dunnen, J. T., Grootscholten, P. M., Bakker, E., Blonden, L. A., Ginjaar, H. B., Wapenaar, M. C., van Paassen, H. M., van Broeckhoven, C., Pearson, P. L., van Ommen, G. J. (1989) Topography of the Duchenne muscular dystrophy (DMD) gene: FIGE and cDNA analysis of 194 cases reveals 115 deletions and 13 duplications. Am. J. Hum. Genet., 45, 885-847.
4. Koenig, M., Beggs, A. H., Moyer, M., Scherpf, S., Heindrich, K., Bettecken, T., Meng, G., Muller, C. R., Lindlof, M., Kaariainen, H., et al. (1989) The molecular basis for Duchenne versus Becker muscular dystrophy: correlation of severity with type of deletion. Am. J. Hum. Genet., 45, 498-506.
5. Tuffery-Giraud, S., Chambert, S., Demaille, J., Claustres, M. (1999) Point mutations in the dystrophin gene: evidence for frequent use of cryptic splice sites as a result of splicing defects. Hum. Mutat., 14, 359-368.
6. Prior, T. W., Bartolo, C., Pearl, D. E, Papp, A. C., Snyder, P. J., Sedra, M. S., Burghes, A. H., Mendell, J. R. (1995) Spectrum of small mutations in the dystrophin coding region. Am. J. Hum. Genet., 57, 22-33.
7. Moser, H. (1984) Duchenne muscular dystrophy: pathogenetic aspects and genetic prevention. Hum. Genet., 66, 17-40.
8. Emery, A. E. (2002) The muscular dystrophies. Lancet, 359, 687-695.
9. Yoshida, M., Ozawa, E. (1990) Glycoprotein complex anchoring dystrophin to sarcolemma. J. Biochem. Tokyo), 108, 748-752.
10. Ervasti, J. M., Campbell, K. P. (1991) Membrane organization of the dystrophin-glycoprotein complex. Cell 66, 1121-1181.
11. Di Blasi C., Morandi, L., Barresi, R., Blasevich, F., Cornelio, F., Mora, M. (1996) Dystrophin-associated protein abnormalities in dystrophin-deficient muscle fibers from symptomatic and asymptomatic Duchenne/Becker muscular dystrophy carriers. Acta Neuropathol. (Berl), 92, 369-377.
12. Ervasti J. M., Ohlendieck, K, Kahl, S. D., Gaver, M. G., Campbell K. P. (1990) Deficiency of a glycoprotein component of the dystrophin complex in dystrophic muscle. Nature, 345, 315-319.
13. Matsumura, K, Burghes, A. H., Mora, M., Tome, F. M., Morandi, L., Cornello, F., Leturcq, F., Jeanpierre, M., Kaplan, J. C., Reinert, P., et al. (1994) Immunohistochemical analysis of dystrophin-associated proteins in Becker/Duchenne muscular dystrophy with huge in-frame deletions in the NH2-terminal and rod domains of dystrophin. J. Clin. Invest., 93, 99-105.
14. Monaco, A. P., Bertelson, C. J., Liechti-Gaaati, S., Moser, H., Kutkel, L. M. (1988) An explanation for the phenotypic differences between patients bearing partial deletions of the DMD locus. Genomics, 2, 90-95.
15. Clemens, P. R., Duncan, F. J. (2001) Progress in gene therapy for Duchenne muscular dystrophy. Curr. Neurol. Neurosci. Rep., 1, 89-96.
16. Khan, M. A. (1993) Corticosteroid therapy in Duchenne muscular dystrophy. J. Neurol. Sci., 120, 8-14.
17. De Angelis, F. G., Sthandier, O., Berarducci, B., Toso, S., Galluzzi, G., Ricci, E., Cossu, G., Bozzoni, I. (2002) Chimeric snRNA molecules carrying antisense sequences against the splice junctions of exon 51 of the dystrophin pre-mRNA induce exon skipping and restoration of a dystrophin synthesis in Delta 48-50 DMD cells. Proc. Natl. Acad. Sci. USA, 99, 9456-9461.
18. Mann, C. J., Honeyman, K, Cheng, A. J., Ly, T., Lloyd, F., Fletcher, S., Morgan, J. E., Partridge, T. A., Wilton, S. D. (2001) Antisense-induced exon skipping and synthesis of dystrophin in the mdx mouse. Proc. Natl. Acad. Sci. USA, 0.98, 42-47.
19. van Deutekom, J. C., Bremmer-Bout, M., Janson, A. A., Ginjaar, I. B., Baas, F., den Dunnen, J. T., van Ommen, G. J. (2001) Antisense-induced exon skipping restores dystrophin expression in DMD patient derived muscle cells. Hum. Mol. Genet., 10, 1547-1554.
20. Wilton, S. D., Lloyd, F., Carville, E, Fletcher, S., Honeyman, E, Agrawal, S., Kole, R. (1999) Specific removal of the nonsense mutation from the mdx dystrophin mRNA using antisense oligonucleotides. Neuromuscul Disord., 9, 330-838.
21. Dunckley, M. G., Manoharan, M., Villiet, P., Eperon, I. C., Dickson, G. (1998) Modification of splicing in the dystrophin gene in cultured Mdx muscle cells by antisense oligoribonucleotides. Hum. Mol. Genet., 7, 1088-1090.
22. Takeshima, Y., Wada, H., Yagi, M., Ishikawa, Y., Minami, R., Nakamura, H., Matsuo, M. (2001) Oligonucleotides against a splicing enhancer sequence led to dystrophin production in muscle cells from a Duchenne muscular dystrophy patient. Brain Dev., 23, 788-790.
23. Aartsma-Rus, A., Bremmer-Bout, M., Janson, A., den Dunnen, J., van Ommen, G., van Deutekom, J. (2002) Targeted exon skipping as a potential gene correction therapy for Duchenne muscular dystrophy. Neuromuscul. Disord., 12, S71.
24. Aartsma-Rus, A. et al “Targeted exon skipping as a potential gene correction therapy for Duchenne muscular dystropby.” Neuromuscul Disord 12.Suppl (2002): S71-S77.
25. Aartsma-Rus, A. et al. “Therapeutic antisense-induced exon shipping in cultured muscle cells from six different DMD patients.” Hum Mol Genet. 12.8 (2003): 907-14.
26. Aartsma-Rus₃, A. et al. “Antisense-induced multiexon skipping for duchenne muscular dystrophy makes more sense.” Am J Hum Genet 74.1 (2004): 83-92.
27. Braasch, D. A. & Corey, D. R. Novel antisense and peptide nucleic acid strategies for controlling gene expression. Biochemistry 41, 4503-10. (2002).
28. Cartegni, L., S. L. Chew, and A. R. Krainer. “Listening to silence and understanding nonsense: exonic mutations that affect splicing.” Nat Rev Genet. 3.4 (2002): 285-98.
29. Cartegni L. et al. “ESEfinder: A web resource to identify exonic splicing enhancers.” Nucleic Acids Reg 31.13 (2003): 3568-71.
30. De Angelis, F. G. et al. “Chimeric snRNA molecules carrying antisense sequences against the splice junctions of exon 51 of the dystrophin pre-mRNA induce exon skipping and restoration of a dystrophin synthesis in Delta 48-50 DMD cells.” Proc Natl Acad Sci USA 99.14 (2002): 9456-61.
31. Dunckley, M. G. et al. “Modification of splicing in the dystrophin gene in cultured Mdx muscle cells by antisense oligoribonucleotides.” Hum Mol Genet. 7.7 (1998): 1083-90.
32. Elayadi, A. N. & Corey, D. R. Application of PNA and LNA oligomers to chemotherapy. Curr Opin Investig Drugs 2, 558-61. (2001).
33. Ginjaar, I. B. et al. “Dystrophin nonsense mutation induces different levels of exon 29 skipping and leads to variable phenotypes within one BMD family [In Process Citation].” Eur J Hum Genet. 8.10 (2000): 798-6.
34. Goyenvalle, A. et al. “Rescue of dystrophic muscle through U7 snRNA-mediated exon-skipping.” Science 306.5702 (2004): 1796-99.
36. Hothorn, T and Hornik, K. Exact Distributions for Rank and Permutation Tests. [0.8-9]. 19-10-2004.
36. Larsen, H. J., Bentin, T. & Nielsen, P. E. Antisense properties of peptide nucleic acid. Biochim Biophys Acta 1489, 159-66. (1999).
37. Lu, Q. L. et al. “Functional amounts of dystrophin produced by skipping the mutated exon in the mdx dystrophic mouse.” Nat Med 6 (2003): 6.
38. Lu, Q. L. et al. “Systemic delivery of antisense oligoribonucleotide restores dystrophin expression in body-wide skeletal muscles.” Proc. Natl Acad. Sci. U.S.A. 102.1 (2005): 198-208.
39. Mann, C. J. et al “Antisense-induced exon skipping and synthesis of dystrophin in the mdx mouse.” Proc Natl Acad Sci USA 98.1 (2001): 42-7.
40. Mann, C. J. et al. “Improved antisense oligonucleotide induced exon skipping in the mdx mouse model of muscular dystrophy.” J Gene Med 4.6 (2002): 644-54.
41. Mathews, D. H. et al. “Expanded sequence dependence of thermodynamic parameters improves prediction of RNA secondary structure.” J Mol Biol 288.5 (1999): 911-40.
42. Monaco, A. P. et al. “An explanation for the phenotypic differences between patients bearing partial deletions of the DMD locus.” Genomics 2.1 (1988): 90-5.
43. R Development Core Team. R: A language and environment for statistical computing. [2.0.1]. 2005
44. \check{S}misterová, J., Wagenaar, A., Stuart, M. C. A., Polushidn, E., ten Brinke, G., Rulst, R., Engberts, J. B. F. N., Hoekstra, D., ‘Molecular shape of the Cationic Lipid Controls the Structure of the Cationic Lipid/Dioleylphosphatidylethanolamine-DNA Complexes and the Efficiency of Gene Delivery’, J. Biol. Chem. 2001, 276, 47615
45. Summerton, J. & Weller, D. Morpholino antisense oligomers: design, preparation, and properties. Antisense Nucleic Acid Drug Dev 7, 187-95 (1997)
46. Takeshima, Y. et al. “Modulation of in vitro splicing of the upstream intron by modifying an intra-exon sequence which is deleted from the dystrophin gene in dystrophin Kobe.” J Clin Invest 95.2 (1995): 515-20.
47. Takeshima, Y. et al. “Oligonucleotides against a splicing enhancer sequence led to dystrophin production in muscle cells from a Duchenne muscular dystrophy patient.” Brain Dev 23.8 (2001): 788-90.
48. van Deutekom, J. C. et al. “Antisense-induced exon skipping restores dystrophin expression in DMD patient derived muscle cells.” Hum Mol Genet. 10.15 (2001): 1547-54.
49. Wahlestedt, C. et al. Potent and non-toxic antisense oligonucleotides containing locked nucleic acids. Proc. Nat. Acad Sci USA 97, 5688-8. (2000).
50. Wilton, S. D. et al. “Specific removal of the nonsense mutation from the mdx dystrophin mRNA using antisense oligonucleotides.” Neuromuscul Disord 9.5 (1999): 330-8.
51. WO 2004/083432

1. A method for generating an oligonucleotide, the method comprising:
determining a binding site or putative binding site for an SR (Ser-Arg) protein in RNA of an exon, and
producing an oligonucleotide that is complementary to said RNA and that at least partially overlaps said binding site or putative binding site.

2. The method according to claim 1, further comprising
determining from a secondary structure of said RNA, a region that is hybridized to another part of said RNA (closed structure) and a region that is not hybridized in said structure (open structure), and
subsequently generating an oligonucleotide that at least partially overlaps said binding site or putative binding site and that overlaps at least part of said closed structure and overlaps at least part of said open structure.

3. The method according to claim 2, wherein said open and closed structures are adjacent to each other.

4. The method according to claim 1, wherein said oligonucleotide is complementary to a consecutive part of between 14 and 50 nucleotides of said RNA.

5. The method according to claim 1, wherein said oligonucleotide comprises RNA.

6. The method according to claim 1, wherein said oligonucleotide is 2′-O-methyl RNA and has a full-length phosphorothioate backbone.

7. The method according to claim 1, wherein pre-mRNA comprising said exon exhibits undesirable splicing in a subject.

8. The method according to claim 7, wherein the absence of said exon from mRNA produced from said pre-mRNA, generates a coding region for a protein.

9. The method according to claim 7, wherein the gene from which said RNA comprising said exon is transcribed, encodes an aberrant Duchenne muscular dystrophy gene (DMD), a collagen VI alpha 1 gene (COL6A1), a myotubular myopathy 1 gene (MTM1), a dysferlin gene (DYSF), a laminin-alpha 2 gene (LAMA2), an Emery-Dreyfus muscular dystrophy gene (EMD), and/or a calpain 3 gene (CAPN3).

10. The method according to claim 9, wherein said gene is the Duchenne muscular dystrophy gene.

11. The method according to claim 1, wherein said SR protein is SF2/ASF or SC35 or SRp40 .

12. The method according to claim 10, wherein said exon comprises exon 8, 46, 48, 52, 54-56, 58, 60-63 or 71-78.

13. An oligonucleotide generated by the method according to claim 1.

14. An oligonucleotide comprising the sequence as depicted in Table 2 or an equivalent thereof.

15. A method for at least partially altering recognition of an exon in a pre-mRNA, the method comprising:
interacting the pre-mRNA with the oligonucleotide of claim 13 so as to at least in part alter recognition of the exon.

16. (canceled)

17. (canceled)

18. (canceled)

19. The method according to claim 15, wherein exon skipping is induced in the pre-mRNA.

20. A method for altering exon-recognition in a pre-mRNA, the method comprising:
interacting the pre-mRNA with the oligonucleotide or equivalent thereof of claim 14, so as to alter exon-recognition in the pre-mRNA

21. A method for altering the efficiency with which an exon in a pre-mRNA is recognized by a splicing machinery, said pre-mRNA being encoded by a gene comprising at least two exons and at least one intron, said method comprising:
providing a transcription system comprising said splicing machinery and said gene, with a first oligonucleotide selected from the group consisting of an oligonucleotide comprising the sequence as depicted in Table 2 and an oligonucleotide generated by a method comprising determining a binding site or putative binding site for an SR (Ser-Arg) protein in RNA of an exon and producing an oligonucleotide that is complementary to said RNA and that at least partially overlaps said binding site or putative binding site, wherein said first oligonucleotide is capable of hybridizing to at least one of said exons, and
allowing for transcription and splicing to occur in said transcription system.

22. The method according to claim 21, wherein said gene comprises at least 3 exons.

23. The method according to claim 21, further comprising:
providing said transcription system with at least a second oligonucleotide capable of hybridizing to at least another of said exons.

24. The method according to claim 23, wherein said first oligonucleotide and said second oligonucleotide are physically linked to each other.
